(19) United States
(12) Patent Application Publication
(10) Pub. No.: US 2003/0037591 A1
(43) Pub. Date: Feb. 27, 2003

(54) Vessel prosthesis with a measuring point

(75) Inventors: Tim Ashton, Ayrshire (GB); Erich Klaeui, Seuzach (CH); Urs Moser, Zurich (CH); Felix Hirt, Wila (CH)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sulzer Markets and Technology Ltd., Winterthur (CH)

(21) Appl. No.: 10/188,375

(22) Filed: Jul. 1, 2002

(30) Foreign Application Priority Data
Jul. 4, 2001 (EP) 01810650.0

(51) Int. Cl. G01M 7/00; A61F 2/06

(52) U.S. Cl. 73/12.08; 623/912; 623/1.1

(57) ABSTRACT

The combination of a human medical vessel prosthesis (1) comprising a measuring probe (2) with a transponder, which measuring probe can be anchored to the vessel prosthesis, and allows the function of the vessel prosthesis to be monitored over longer periods of time. Suitable measurement parameters such as pressure values can be detected by the measuring probe (2) and be transmitted in a wireless manner with the aid of the transponder to a transceiver apparatus where they are available for evaluation.

The invention relates to a vessel prosthesis having a measuring probe with a transponder, wherein the measuring probe can be anchored to the vessel prosthesis and is suitable to detect measurement parameters by means of which the function of the vessel prosthesis can be checked, and wherein the transponder is formed to interact with a transceiver apparatus.

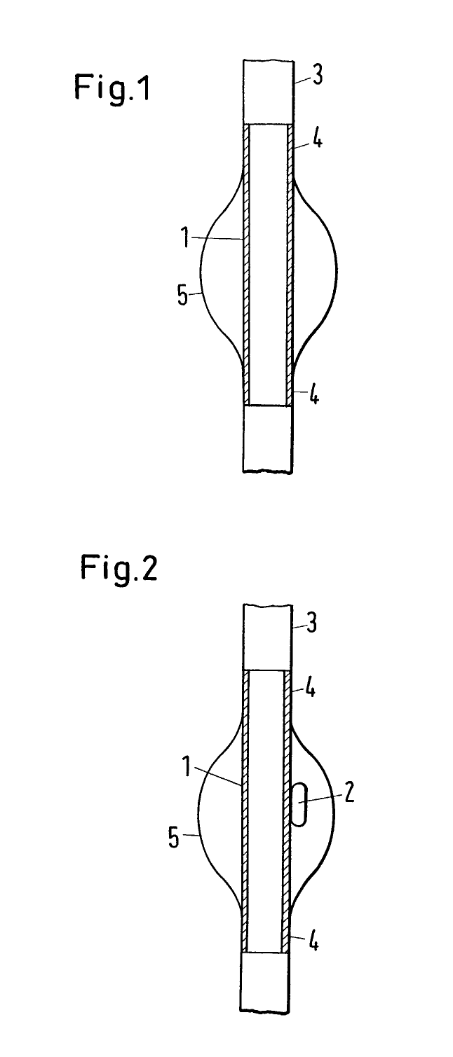

Vessel prostheses are today used in human medicine in the treatment of vascular injuries, vascular diseases and vascular anomalies. The area of use includes not only the intravascular area, but also, for example, the trachea, the digestive tract and the urinary tracts. As a typical example of a vessel prosthesis, the product Anaconda™ of the company of Sulzer Vascutek Ltd. can be mentioned which is used in the treatment of blood vessel dilatations (so-called aneurysms). The vessel prosthesis is inserted into the affected vessel in order to isolate the expanded point and to relieve the pressure and thus to prevent a further expansion and the breaking of the relevant vessel. FIG. 1 shows such a vessel prosthesis in the implanted state. The vessel prosthesis consists essentially of a piece of hose 1 which was inserted into the blood vessel 3 to insulate the sack-shaped vaso-dilatation. With an intact vessel prosthesis, the transitions 4 from the hose piece 1 to the vessel wall is completely sealed at both sides of the vaso-dilatation 5 so that no blood can enter into the vaso-dilatation 5 from the blood vessel 3. The vaso-dilatation 5 is thus relieved of the pressure prevailing in the vessel 3 and does not further expand. In the event of a leak at the transitions, however, blood enters into the vaso-dilatation 5, whereupon this expands further until it tears. If this process is not discovered and interrupted in time, this can be fatal for the patient. The proper function of the vessel prosthesis, that is in this case the tightness of the vessel prosthesis 1 and of the transitions 4, is of decisive importance for the patient. A suitable monitoring system, which allows the tightness of the implanted vessel prosthesis to be monitored periodically or continuously, is, however, lacking in the prior art. A measuring probe is admittedly described in the document WO 99/59467 which is fixed at the end of a catheter and with which the state parameters and measurement parameters such as temperature, pressure, etc. can be locally detected at the inside of the body. However, it is generally not possible with the measuring probe described in the document WO 99/59467 to carry out measurements for checking the function of implanted vessel prostheses without using a surgical procedure. A further disadvantage of the measuring probe described in the document WO 99/59467 consists of the fact that the measuring probe has to be separately introduced and positioned at the measuring point for the measurement, which means additional cost and complexity.

It is the underlying object of the invention to make available a system for the monitoring of the function of vessel prostheses while avoiding the disadvantages known from the prior art. In particular, the system should be suitable to detect leaks which are beginning at an early time.

This object is satisfied by the subject matter of the invention as defined in the independent claims.

The vessel prosthesis of the invention comprises a measuring probe with a transponder which can be anchored at the vessel prosthesis, the measuring probe being suitable to detect measurement parameters by means of which the function of the vessel prosthesis can be checked, and the transponder being formed to interact with a transceiver apparatus, for example a so-called interrogator. Measurement parameters by means of which the functioning of the vessel prosthesis can be checked are, for example, mechanical parameters such as volume or shape changes, forces, pressures or the like, or biological parameters such as pH values, concentrations of electrolytes, of blood gas or of protein or similar. The vessel prosthesis of the invention allows changes in connection with the implanted vessel prosthesis, such as starting leak which is beginning, to be detected at an early time by periodic, for example quarterly, detection and long term evaluation of the said measurement parameters.

The measuring probe is preferably mechanically connected to the vessel prosthesis or integrated in the vessel prosthesis. In a preferred embodiment, the measuring probe is fastened to the surface of the vessel implant by means of fastening lines.

The vessel prosthesis is preferably flexible and can be implanted together with the measuring probe via a body vessel. For this purpose, for example, the folded up vessel prosthesis and the measuring probe anchored to the same is inserted into a catheter and the catheter prepared in this way is introduced into a body vessel and placed at the position provided for the implantation. The vessel prosthesis and the measuring probe anchored to the same is pushed out of the catheter with the aid of a control rod, with the vessel prosthesis being unfolded up to the full length and the measuring probe being brought into its end position. The method of the invention has the advantage that a separate implantation of the measuring probe is omitted. The measuring probe can furthermore be positioned in a simple manner with the method of the invention at places which are only surgically accessible after the insertion of the vessel prosthesis such as the outer surface of the vessel prosthesis.

The vessel prosthesis is preferably substantially formed in a hose-like manner and/or replicated in the shape of a vessel or a part of a vessel in order to insulate a pathological vaso-dilatation from the inside in the implanted state, with the measuring probe being provided on the outer surface of the vessel prosthesis so that the sealing of the vessel prosthesis can be monitored with respect to the vaso-dilatation.

In a preferred embodiment, the measuring probe comprises a sleeve and a pressure sensor which is arranged in the interior of the sleeve, with the sleeve being filled with a pressure-transmitting medium. The sleeve is preferably elastic so that the whole surface of the sleeve acts as a pressure absorber in the pressure measurement. The deformation required for the pressure measurement is minimised thereby, which is particularly advantageous if deposits have formed on the measuring probe.

The transponder is preferably formed as a passive transponder. A passive transponder is an electrical transmission apparatus for the wireless transmission of measured values without its own separate power supply.

The pressure sensor is preferably integrated into the passive transponder circuit, for example by the transponder including a capacitive pressure sensor and an inductor, with the capacitive pressure sensor and the inductor being mutually connected such that they form a resonant circuit. Alternatively, the passive transponder can also include an inductive pressure sensor and a capacitor which together form a resonant circuit. Such a transponder can be realised with minimum effort. The passive transponder preferably additionally includes a non-linear component, for example a capacitive diode. Harmonics are produced by the non-linear component such that a measured value to be transmitted is transmitted at a frequency which is different from the frequency of the interrogator radiation with which the transponder is excited. In this way, the transponder signal can be better separated from the interrogator radiation at the receiver side.

A transceiver apparatus, which is especially suitable for the interaction with the transponder of the vessel prosthesis of the invention, is the subject of claim 12. The transceiver apparatus comprises an interrogator antenna which is designed to surround the body part in which the vessel prosthesis is implanted, for example in the stomach. The complete surrounding of the body part by the interrogator antenna has the advantage that a better coupling is achieved between the interrogator antenna and the transponder when the vessel prosthesis and the associated transponder are located deep inside the body. The interrogator antenna is preferably arranged in a belt-like apparatus. The interrogator antenna preferably comprises at least one primary antenna and one secondary antenna, with the two antennas being coupled such that the far-field of the coupled antennas is attenuated. This antenna arrangement has the advantage that interference of ambient instruments and radio services by the relatively strong interrogator radiation can be avoided without the wireless energy supply of the transponder being impaired.

Further advantageous embodiments of the invention can be found in the dependent claims and the drawing.

The invention is explained in more detail in the following with reference to the embodiments and to the drawing. There are shown:

FIG. 1: a longitudinal section through a conventional vessel prosthesis in the implanted state;

FIG. 2: a longitudinal section through a vessel prosthesis in accordance with a first embodiment of the present invention in the implanted state;

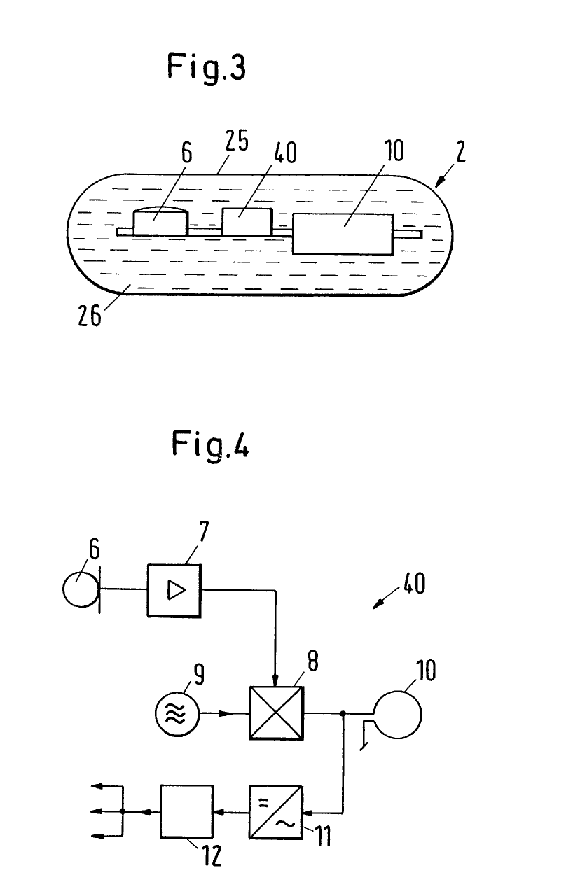

FIG. 3 a section through a measuring probe in accordance with the first embodiment of the present invention;

FIG. 4 a block diagram of a passive transponder in accordance with the first embodiment of the present invention;

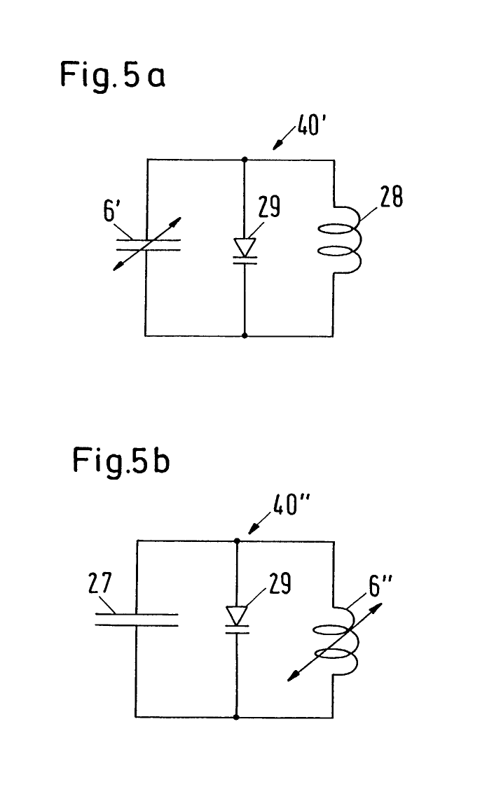

FIG. 5a a circuit diagram of a variant of a passive transponder with an integrated capacitive pressure sensor;

FIG. 5b a circuit diagram of a variant of a passive transponder with an integrated inductive pressure sensor;

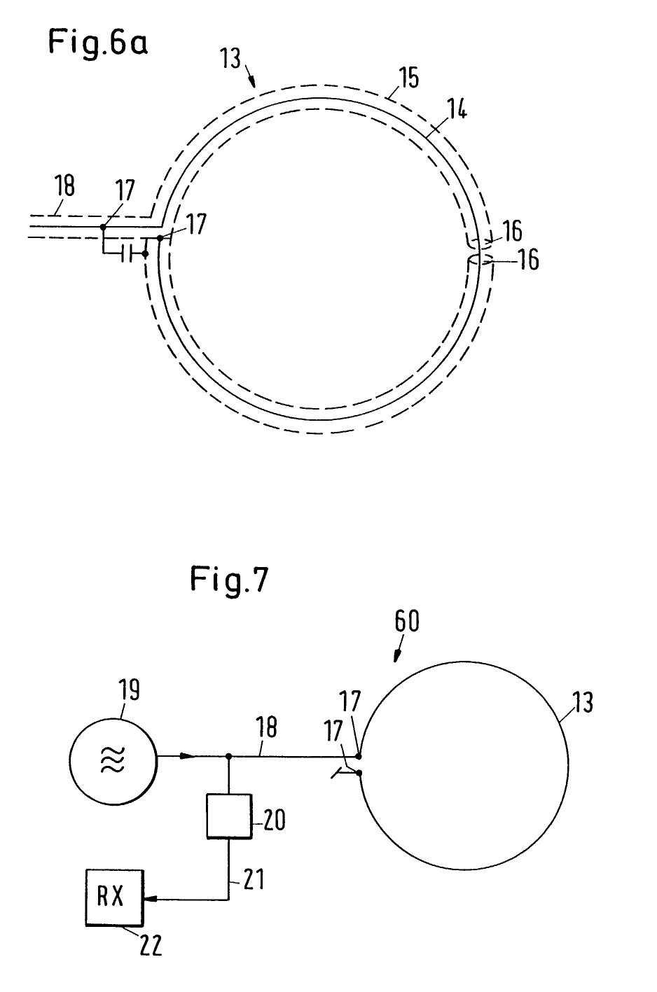

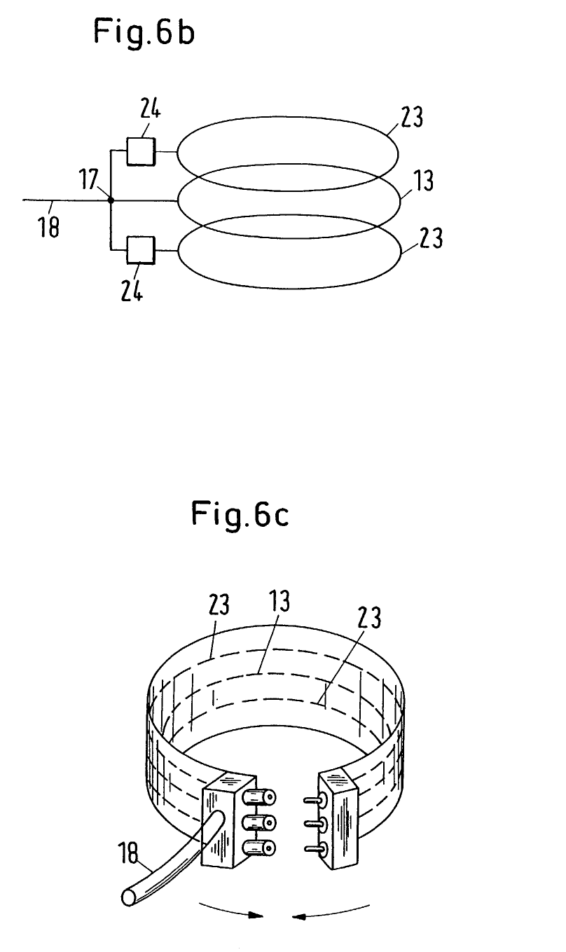

FIG. 6a an interrogator antenna in accordance with the first embodiment of the present invention;

FIG. 6b an interrogator antenna with one primary antenna and two secondary antennas;

FIG. 6c the constructional design of the interrogator antenna of FIG. 6b;

FIG. 7 a block diagram of an interrogator in accordance with the first embodiment of the present invention;

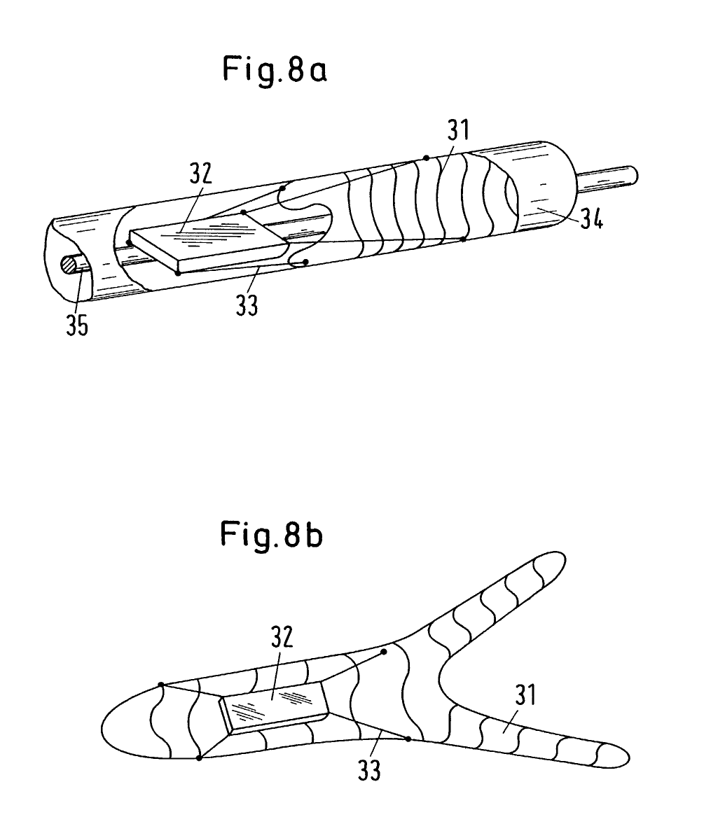

FIG. 8a a vessel prosthesis in accordance with a second embodiment of the present invention during the insertion phase;

FIG. 8b the vessel prosthesis in accordance with the second embodiment in the completely unfolded state;

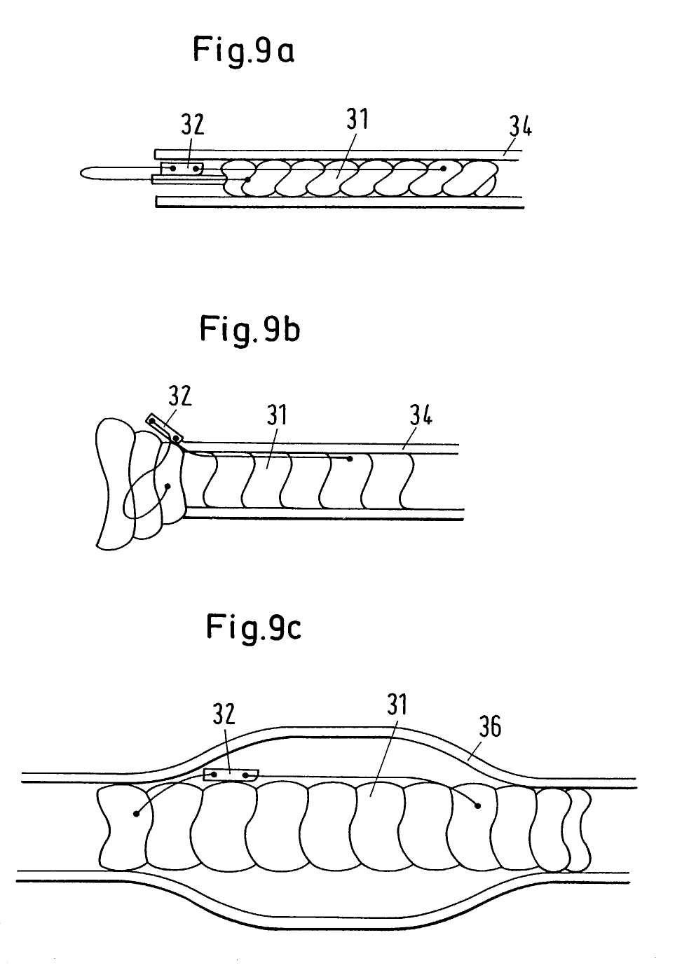

FIG. 9a a longitudinal section through the vessel prosthesis in accordance with the second embodiment folded up inside a catheter;

FIG. 9b a longitudinal section through the vessel prosthesis in accordance with the second embodiment during the unfolding;

FIG. 9c a longitudinal section through the vessel prosthesis in accordance with the second embodiment completely unfolded inside an aneurysm.

A vessel prosthesis in accordance with a first embodiment of the present invention is illustrated in FIG. 2. The vessel prosthesis comprises a hose piece 1, which was inserted into the blood vessel 3 to insulate the sack-shaped vaso-dilatation 5, and a measuring probe 2 anchored to the hose piece 1. With an intact vessel prosthesis, the transitions 4 from the hose piece 1 to the vessel wall are completely sealed at both sides of the vaso-dilatation 5 so that no blood can enter into the vaso-dilatation 5 from the blood vessel 3. The vaso-dilatation 5 is thus relieved of the pressure prevailing in the vessel 3 and does not expand further. The measuring probe 2 is anchored at the outer surface of the hose piece 1 in order to monitor the sealing of the hose piece 1 with respect to the vaso-dilatation 5.

FIG. 3 shows a measuring probe 2 in accordance with the first embodiment of the present invention. The measuring probe 2 comprises an elastic sleeve 25, a pressure sensor 6 to measure the pressure in the enclosed volume between the hose piece 1 and the vaso-dilatation 5 and a passive transponder 40 with an antenna 10 to transmit the measured pressure values. The pressure sensor 6 is arranged in the interior of the sleeve 25 and the sleeve 25 is filled with a pressure-transmitting medium 26, for example an oil or a gel, which transmits the pressure acting on the sleeve 25 to the pressure sensor 6. This arrangement is particularly advantageous if deposits have formed on the measuring probe since the deformations of the sleeve required for the pressure measurement are minimal. The pressure sensor 6 can, however, also be arranged externally and connected to the transponder 40 by a cable or be integrated in the measuring probe sleeve 25. The measurement principle of the pressure sensor can be of a piezo-resistive kind, a capacitive kind, an inductive kind, a magneto-elastic kind, etc.

In the embodiment, the pressure sensor is connected to a passive transponder. A passive transponder is an electronic transmission apparatus for the wireless transmission of measured values without its own power supply. The transponder is irradiated with high frequency radiation for its activation by a transceiver apparatus, also called an interrogator in the following, whereupon the transponder in turn emits a high frequency carrier signal which is modulated with the information to be transmitted. This transponder signal can be received in the receiver of the transceiver apparatus and demodulated for the purpose of obtaining the transmitted information. The energy supply of the measuring circuit likewise expediently takes place from the radiation energy picked up by the transponder.

FIG. 4 shows a block circuit diagram of a passive transponder 40 in accordance with a first embodiment of the present invention, The transponder 40 is supplied with energy by the high frequency voltage field emitted by an interrogator. The radiation induces a high frequency radiation in an antenna 10 which is rectified in a rectifier 11 and supplied to a feed module 12. In the feed module 12 there are located a storage capacitor, which is charged with the direct current supplied by the rectifier, and switches which switch on the transponder 40 when the voltage at the capacitor has reached the required operating voltage and which switch off the transponder 40 again when the minimum operating voltage is fallen below. This kind of energy supply is a sequential mode of operation. The sequential mode of operation has the advantage that a substantially weaker radiation field is required than with duplex operation where the interrogator and the transponder are active simultaneously, since the transmission phase as a rule only takes fractions of seconds, whereas a plurality of seconds is available for the charging phase in sequential operation. The measured value fed to the pressure sensor 6 is processed in a signal processing module 7 where it can, for example, be converted into a frequency modulated auxiliary carrier or digitised. The signal processed in this way is fed into a modulator 8 where it modulates the carrier produced in an oscillator 9. This modulated carrier is fed into the antenna 10 and radiated from there as a transponder signal. The carrier signal can be also be gained by reflection or frequency multiplication or division from the interrogator radiation instead of by the oscillator 9. A modulation of the antenna impedance of the transponder can also be used instead of a conventional modulator. If the carrier signal is gained by reflection from the interrogator radiation, this results in the so-called load modulation, absorption modulation or backscatter modulation.

In a variant which is shown in FIG. 5a, the pressure sensor is integrated into the passive transponder circuit by the transponder 40′ comprising a capacitive pressure sensor 6′ and an inductor 28, with the capacitive pressure sensor 6′ and the inductor 28 being mutually connected such that they form a resonant circuit. If this resonant circuit is excited by the high frequency radiation of the interrogator, then the resonant circuit simultaneously emits high frequency radiation, with the phase of the emitted radiation changing in dependence on the pressure. The transponder 40″, a shown in FIG. 5b, can alternatively also comprise an inductive pressure sensor 6″ and a capacitor 27, with the inductive pressure sensor 6″ and the capacitor 27 being mutually connected such that they form a resonant circuit. A transponder 40′, 40″ of this kind can be realised with minimum cost and complexity. The passive transponder 40′, 40″ preferably additionally comprises a non-linear component 29, for example a capacitive diode. Harmonics are produced by the non-linear component 29 such that a measured value to be transmitted is transmitted at a frequency which is different to the frequency of the interrogator circuit at which the transponder 40′, 40″ is excited. The transponder signal can thereby be better separated from the interrogator radiation on the receiver side.

FIGS. 6a and 7 show an interrogator antenna 13 and a block diagram of the interrogator 60 in accordance with the first embodiment of the present invention. The interrogator antenna 13 is set up as an electrically shielded loop antenna. It is possible, as a result of the loop design, to surround the body part in which the vessel prosthesis is inserted with the antenna, which has the advantage that a better coupling is achieved between the interrogator antenna and the transponder when the vessel prosthesis and the associated transponder are located deep inside the body. The loop antenna 13 is set up as follows: the actual antenna wire 14 is coaxially located in the interior of a hose-like shield 15 which is interrupted at a point 16. The interrupted shield ensures that only the magnetic field occurs in the near field of the antenna and not the interfering electrical field. A capacitor is located at the feed point 17 of the loop antenna 13 and the antenna can be tuned to resonance by this. The feed point 17 is connected to a high frequency generator 19 via a shielded cable 18. The transponder signal emitted by the transponder is received by the same antenna 13, is picked up at the feed point 17 and led to a receiver 22 via a frequency separating filter 20 and a cable 21. The frequency separating filter 20 prevents the relatively strong interrogator signal from reaching the receiver input and damaging this.

FIG. 6b shows a preferred variant of an interrogator antenna of the present invention. Compensation loops 23 are attached parallel to the loop antenna 13, symmetrically to the antenna 13 at both sides, at a spacing which corresponds roughly to half the radius to the full radius of the loop antenna 13. The compensation loops 23 are fed by compensation currents whose amplitudes correspond overall roughly to the antenna current in the loop antenna 13 and whose phase is displaced by 180° with respect to the antenna current. The far-field of the loop antenna 13 is largely suppressed thereby without the near-field, which is important for the wireless energy supply, being substantially attenuated. The compensation currents can, for example, be picked up from the feed point 17 of the antenna 13 and supplied to the compensation loops 24 via a matching circuit 24. The amplitude and the phase of the compensation currents can be set with the aid of the matching circuit 24 for an optimum suppression of the far-field. This variant has the advantage that interference of other instruments and radio services by the far-field of the relatively strong interrogator radiation, which is needed for the energy supply of the passive transponder, is largely avoided. In a further variant, the compensation system comprises only one compensation loop 23. In a further variant, the compensation loops 23 are not fed.

The interrogator antenna, which comprises either the antenna 13 alone or the antenna 13 and the compensation system 17, 23 and 24 described in the above variant, is preferably designed as a flexible belt. For the measurement, the belt can be laid around the appropriate body part into which the vessel prosthesis with the associated measuring probe was inserted. An interrogator antenna in the form of a belt is illustrated in FIG. 6c.

FIG. 8a shows a vessel prosthesis in accordance with a second embodiment of the present invention during the insertion phase and FIG. 8b the same vessel prosthesis in the completely unfolded state. In FIG. 8a, the tightly folded vessel prosthesis 31 is inserted into a catheter 34. A measuring probe 32, which is fastened to both ends at the outer surface of the vessel prosthesis 31 with the aid of two fastening lines 33 in each case, is located directly before or after the folded up vessel prosthesis 31. The measuring probe 32 has a slim volume and is equipped with a biocompatible surface. The lengths of the measuring probe 32 and of the rear fastening lines 33 are roughly of the same size. A control rod 35, which serves to push out and unfold the vessel prosthesis 31, extends along the catheter 34.

FIGS. 9a, b and c show the insertion of the vessel prosthesis in accordance with the second embodiment of the present invention into a vasodilatation, a so-called aneurysm. The tightly folded vessel prosthesis 31 is inserted into a catheter 34 together with the measuring probe 32 which is fastened to the vessel prosthesis 31 by means of the fastening lines 33. FIG. 9a shows the vessel prosthesis 31 folded up inside the catheter 34. The catheter 34 prepared in this manner is inserted into a body vessel and placed at the position provided for the implantation, for example at an aneurysm 36. The vessel prosthesis 31 and the measuring probe 32 fastened to the same are pushed out of the catheter 34 and the vessel prosthesis 31 unfolded with the aid of a control rod. FIG. 9b shows the vessel prosthesis 31 during the unfolding. During the unfolding of the vessel prosthesis 31, the measuring probe 32 is pulled onto the outer surface of the vessel prosthesis 31 by the fastening lines. On completion of the unfolding procedure, the vessel prosthesis is unfolded up to the full length and the four fastening lines 33 are stretched such that the measuring probe 32 is fixed on the outer surface of the vessel prosthesis 31. FIG. 9c shows the vessel prosthesis 31 completely unfolded in the aneurysm 36. The method of the invention has the advantage that a separate implantation of the measuring probe is omitted. Furthermore, the measuring probe can be positioned in a simple manner with the method of the invention at points which are only surgically accessible after the insertion of the vessel prosthesis, such as the outer surface of the vessel prosthesis.

1. A vessel prosthesis (1, 31) comprising a measuring probe (2, 32) with a transponder (40, 40′, 40″), wherein the measuring probe can be anchored to the vessel prosthesis and is suitable to detect measurement parameters by means of which the function of the vessel prosthesis (1, 31) can be checked, and wherein the transponder (40, 40′, 40″) is formed to interact with a transceiver apparatus (60).

2. A vessel prosthesis in accordance with claim 1, wherein the measuring probe (2, 32) is mechanically connected to the vessel prosthesis (1, 31) and/or is integrated into the vessel prosthesis (1, 31).

3. A vessel prosthesis in accordance with claim 1 or claim 2, wherein the measuring probe (2, 32) is fastened to the outer surface of the vessel prosthesis (1, 31) by means of at least one fastening line (33).

4. A vessel prosthesis in accordance with any one of claims 1 to 3, wherein the measuring probe (2, 32) which can be anchored to the vessel prosthesis (1, 31) comprises a pressure sensor (6, 6′, 6″).

5. A vessel prosthesis in accordance with any one of claims 1 to 4, wherein the wall of the vessel prosthesis is flexible; and wherein the vessel prosthesis and the measuring probe can be implanted via a body vessel.

6. A vessel prosthesis in accordance with any one of claims 1 to 5, wherein the vessel prosthesis (1, 31) is substantially formed in hoselike manner in order to insulate a pathological vaso-dilatation from the inside in the implanted state; and wherein the measuring probe (2, 32) is provided at the outer surface of the vessel prosthesis (1, 31) such that the tightness of the vessel prosthesis (1, 31) with respect to the dilated vessel can be monitored.

7. A vessel prosthesis in accordance with any one of claims 1 to 6, characterised in that the measuring probe (2, 32) comprises a sleeve (25) and a pressure sensor (6, 6′, 6″) which is arranged in the interior of the sleeve (25); and in that the sleeve (25) is filled with a pressure transmitting medium (26).

8. A vessel prosthesis in accordance with any one of claims 1 to 7, wherein the transponder (40, 40′, 40″) is formed as a passive transponder.

9. A vessel prosthesis in accordance with claim 8, wherein the passive transponder (40′) includes a capacitive pressure sensor (6′) and an inductor (28); and wherein the capacitive pressure sensor (6′) and the inductor (28) are mutually connected such that they form a resonant circuit.

10. A vessel prosthesis in accordance with claim 8, wherein the passive transponder (40″) comprises an inductive pressure sensor (6″) and a capacitor (27); and wherein the inductive pressure sensor (6″) and the capacitor (27) are mutually connected such that they form a resonant circuit.

11. A vessel prosthesis in accordance with any one of claims 8 to 10, wherein the passive transponder (40, 40′, 40″) additionally comprises a non-linear component (29) such that a measured value determined by the measuring probe (2, 32) is transmitted at a frequency which differs from an excitation frequency at which the transponder (40, 40′, 40″) is excited.

12. A transceiver apparatus (60) for interaction with a transponder (40) of a measuring probe (2) which is provided at a vessel prosthesis (1, 31) in accordance with any one of claims 1 to 11, said transceiver apparatus (60) including an interrogator antenna 13, characterised in that the interrogator antenna 13 is formed to surround that body part in which the vessel prosthesis (1, 31) is inserted.

13. A transceiver apparatus in accordance with claim 12, wherein the interrogator antenna 13 is arranged in an apparatus which has the form of a belt.

14. A transceiver apparatus in accordance with claim 12 or claim 13, wherein the interrogator antenna includes at least one primary and at least one secondary antenna; and wherein the two antennas are coupled such that the far-field of the coupled antennas is attenuated.

15. A method for the implantation of a vessel prosthesis (1, 31) having a measuring probe (2, 32) anchorable to the vessel prosthesis (1, 31) in accordance with any one of claims 1 to 11, the method including:
insertion of the folded up vessel prosthesis (1, 31) and of the measuring probe (2, 32) anchored to the same into a catheter (34);
insertion of the catheter (34) into a body vessel;
positioning of the catheter (34) at the point provided for the implantation;
pushing out of the vessel prosthesis (1, 31) and the measuring probe (2, 32) anchored to the same with the aid of a control rod (35), wherein the vessel prosthesis (1, 31) is unfolded to the full length and the measuring probe (2, 32) is brought into its final position.

16. A method in accordance with claim 15, wherein
the measuring probe (2, 32) is fastened to the outer surface of the vessel implant (1, 31) by means of two fastening lines (33) in each case;
the measuring probe (2, 32) is pulled onto the outer surface of the vessel prosthesis (1, 31) by the fastening lines (33) during the unfolding of the vessel prosthesis (1, 31); and
the four fastening lines (33) are stretched in the end state of the unfolding procedure such that the measuring probe (2, 32) is fixed at the outer surface of the vessel prosthesis.
